(19) United States
(12) Patent Application Publication
(10) Pub. No.: US 2009/0131731 A1
(43) Pub. Date: May 21, 2009

(54) Process for the Production of Linear Alpha-Olefins

(75) Inventor: Bryce Alan Willams, Lisle, IL (US)

Correspondence Address: NIXON & VANDERHYE, PC, 901 NORTH GLEBE ROAD, 11TH FLOOR, ARLINGTON, VA, 22203, US

(73) Assignee: INEOS EUROPE LIMITED, Hampshire (GB)

(21) Appl. No.: 11/921,300

(22) PCT Filed: Jun. 28, 2006
(86) PCT No.: PCT/GB2006/002366

§ 371 Date: Dec. 20, 2007

Related U.S. Application Data
(60) Provisional application No. 60/696,850, filed on Jul. 6, 2005.
(60) Provisional application No. 60/696,849, filed on Jul. 6, 2005.

(51) Int. Cl. C07C 4/02

(52) U.S. Cl. 585/650; 585/648

(57) ABSTRACT

The present invention relates to a process for the production of linear alpha-olefins. In particular, the present invention relates to a process for the production of linear alpha-olefins (LAO's) in which Fischer Tropsch (FT)-derived liquids are autothermally cracked to produce linear alpha-olefins or an LAO-containing feed is autothermally cracked to produce a product LAO with a lower number of carbon atoms than the LAO's fed to the process.

The present invention relates to a process for the production of linear alpha-olefins.

Linear alpha-olefins (LAO's) may be generally produced by the oligomerisation of ethylene, as described, for example, in US 2004/0122271 A1.

The products formed generally comprise a distribution of LAO's of general formula C₂ₙH₍₄ₙ₊₁₎CH═CH₂, where n is 1, 2, 3, etc., i.e. 1-butene, 1-hexene, 1-octene etc.

LAO's may be used as surfactants and lubricants, but the most valuable uses of the “lower” LAO's, especially 1-hexene and 1-octene, if they can be obtained in high enough purity, are as co-monomers for polymer production.

The “higher” LAO's are generally less valuable.

(“Lower” LAO and “higher” LAO as used herein refer to the relative number of carbon atoms in the respective LAO's.)

It is, however, difficult to target specific LAO's by ethylene oligomerisation due to the inherent product distribution obtained, and hence, significant proportions of higher LAO's are obtained. A typical product distribution may contain approximately 70% C10 and lower LAO's, 20% C12 and C14, 5% C16 and C18 and 5% C20 and above.

It is desired therefore to find a process by which the more valuable LAO's may be obtained.

It has also been disclosed that linear alpha-olefins may be produced by autothermal cracking of paraffinic hydrocarbons, as described in US2004/0199038.

Autothermal cracking is a route to olefins in which a hydrocarbon feed is mixed with oxygen and passed over an autothermal cracking catalyst. The autothermal cracking catalyst is capable of supporting combustion beyond the fuel rich limit of flammability. In autothermal cracking, combustion is initiated on the catalyst surface and the heat required to raise the reactants to the process temperature and to carry out the endothermic cracking process is generated in situ. The autothermal cracking of paraffinic hydrocarbons is described in EP-332289B; EP-529793B; EP-709446A and WO 00/14035.

More recently, it has been found that unsaturated hydrocarbons may be co-fed to an autothermal cracking process, as described in WO 2004/087626.

It has now been found that LAO's of high purity may be advantageously obtained by autothermally cracking a hydrocarbon feedstream which has been selected to produce high yields of LAO's whilst enabling relatively easy separation of the desired LAO's in high purity.

Hence, in a first aspect, the present invention provides a process for the production of linear alpha-olefins, which process comprises:

(i) providing a liquid hydrocarbon stream comprising hydrocarbons having at least N carbon atoms, wherein

a) the liquid hydrocarbon stream comprises linear paraffinic hydrocarbons, N is at least 9, and the liquid hydrocarbon stream has been derived from the product stream of a Fischer-Tropsch process by separation of hydrocarbons having less than N carbon atoms therefrom, or

b) the liquid hydrocarbon stream is an LAO-containing stream comprising LAO's having at least N carbon atoms, where N is at least 10,

(ii) autothermally cracking the liquid hydrocarbon stream to produce an ATC product stream comprising linear alpha-olefins having M or less carbon atoms, where M is less than N, and
(iii) separating the linear alpha olefins having M or less carbon atoms from the ATC product stream.

Use of a liquid hydrocarbon stream comprising hydrocarbons with more than M carbon atoms as the stream to be cracked has the advantage that the desired LAO's (having M or less carbon atoms), can be relatively easily separated from unreacted hydrocarbons in the ATC product stream, for example by distillation. Unreacted hydrocarbons, can be recycled to the autothermal cracking step and cracked to improve the yield of the desired LAO's.

The ease of separation allows the desired LAO's to be produced with high purity using relatively simple separation steps, such as distillation.

In a first embodiment, it has been found that autothermal cracking can be applied to an LAO-containing feed to produce LAO's with a lower number of carbon atoms than the LAO's fed to the process. Hence, autothermal cracking may be used to upgrade the lower value, higher LAO's to produce more valuable, lower LAO's.

In this embodiment, step (i) of the process of the present invention comprises providing an LAO-containing stream comprising LAO's having at least N carbon atoms, where N is at least 10.

Preferably, the LAO-containing stream is derived from a process for the production of LAO's by oligomerisation of olefins, especially ethylene. Thus, the LAO-containing stream may comprise a portion of the product stream of an LAO oligomerisation process, said portion comprising LAO's having at least N carbon atoms (where N is at least 10). In particular, the LAO-containing stream comprising LAO's having at least N carbon atoms,

where N is at least 10, may be obtained by separating the required components of the LAO process product stream having at least N carbon atoms from the full LAO process product stream. This separation may be achieved by any suitable means, for example by distillation. Product streams of LAO's having less than N carbon atoms, such as 1-hexene and 1-octene, may be recovered from the LAO process product stream using conventional LAO process separation and purification techniques.

Hence, autothermal cracking may be used to upgrade the lower value, higher LAO's produced from a conventional LAO process to produce more valuable LAO's.

Preferably, the LAO-containing stream comprises essentially no LAO's with less than N carbon atoms, for example, less than 5 mol %, especially less than 1 mol %, of LAO's with less than N carbon atoms.

In a second, preferred, embodiment of the present invention step (i) of the process of the present invention comprises providing a liquid hydrocarbon stream comprising linear paraffinic hydrocarbons having at least N carbon atoms, where N is at least 9, which liquid hydrocarbon stream has been derived from the product stream of a Fischer-Tropsch (FT) process by separation of hydrocarbons having less than N carbon atoms therefrom. The Fischer-Tropsch process produces hydrocarbons from carbon monoxide and hydrogen (synthesis gas). Typically the product stream from a Fischer-Tropsch reactor includes C4 to C20+ hydrocarbons. The hydrocarbons are generally highly linear. Depending on the catalyst and process used, the product stream may be highly paraffinic or may comprise a substantial proportion of olefins.

Thus, in this embodiment step (i) generally comprises providing an FT reactor effluent comprising C4 to C20+ hydrocarbons and treating said effluent to remove hydrocarbons with less than N carbon atoms to provide said liquid hydrocarbon stream comprising linear paraffinic hydrocarbons having at least N carbon atoms.

The FT reactor effluent (comprising C4 to C20+ hydrocarbons) may be treated by any suitable technique, for example by distillation, to remove hydrocarbons with less than N carbon atoms and to give a liquid hydrocarbon stream comprising linear paraffinic hydrocarbons with at least N carbon atoms.

Preferably, the FT reactor effluent may be treated and subsequently passed to the autothermal cracking step without treatment to reduce the olefin content thereof.

Product streams of linear paraffinic hydrocarbons having less than N carbon atoms may be recovered from the FT process product stream using conventional process separation and purification techniques.

In general, the most desired LAO product, and hence the preferred feed (preferred value of N) may be determined based on the relative values of the LAO monomers, which may vary with market conditions.

For example, in the second embodiment, if it is desired to produce 1-decene (C10, M=10), a liquid hydrocarbon stream comprising linear paraffinic hydrocarbons having at least 11 carbon atoms (C11, N=11) will form the feed. As well as 1-decene, “lower” LAO's, such as 1-octene (C8), 1-hexene (C6) and 1-butene (C4) may also be produced.

Alternatively, where 1-octene is the most valuable/desired LAO, a liquid hydrocarbon stream comprising linear paraffinic hydrocarbons having at least 9 carbon atoms may form the feed.

Although the above describe use of a liquid hydrocarbon stream comprising linear paraffinic hydrocarbons having at least N carbon atoms where N=M+1 for the second embodiment, it may be preferred to use a liquid hydrocarbon stream comprising linear paraffinic hydrocarbons having at least N carbon atoms, wherein N>M+1, for example, N=M+2, since this will further ease the subsequent separation of the product LAO's from unreacted linear paraffinic hydrocarbons.

Although small quantities of hydrocarbons with less than N carbon atoms may be tolerated in the liquid hydrocarbon stream, preferably, the liquid hydrocarbon stream comprises essentially no hydrocarbons with less than N carbon atoms, for example less than 5 mol %, especially less than 1 mol %, of hydrocarbons with less than N carbon atoms.

In step (ii) of the process of the present invention, the liquid hydrocarbon stream is autothermally cracked to produce an ATC product stream comprising LAO's having M or less carbon atoms, where M is less than N. In particular, the liquid hydrocarbon stream is autothermally cracked by contacting said stream with a catalyst capable of supporting combustion beyond the fuel rich limit of flammability in the presence of an oxygen-containing gas.

The ATC product stream is quenched as it emerges from the reaction chamber to avoid further reactions taking place. Usually the product stream is cooled to between 750-600° C. within less than 100 milliseconds of formation, preferably within 50 milliseconds of formation and most preferably within 20 milliseconds of formation e.g. within 10 milliseconds of formation. The heat from the quenching may be used to generate high-pressure steam, which can be used to provide power for those parts of the overall process requiring it.

The ATC product stream may also comprise ethylene.

In the first embodiment of the process of the present invention, the ethylene in the ATC product stream is preferably separated and passed to the LAO process from which the LAO-containing stream is provided. In this embodiment, the LAO process upgrades ethylene to LAO's and any higher LAO's produced are cracked back down to ethylene and lower LAO's in the autothermal cracker. Thus, the process produces improved yields of the most valuable LAO's, such as 1-octene, 1-hexene, and where N is greater than 10, 1-decene.

In addition to olefins, the autothermal cracking reaction produces hydrogen, carbon monoxide, methane, and small amounts of acetylenes, aromatics and carbon dioxide.

In step (iii) of the process of the present invention, the LAO's having M or less carbon atoms are separated from the ATC product stream. This may be achieved by any suitable technique or series of techniques. For example, an amine wash may be used to remove carbon dioxide and water from the ATC product stream, a demethaniser to remove hydrogen, carbon monoxide and methane, and hydrogenation to remove acetylenic compounds and dienes. Distillation is a particularly suitable technique for separation of the desired LAO's in the product stream, especially due to the different carbon numbers of the desired LAO products from any unreacted feed present.

The unreacted feed is generally separated as a stream comprising said unreacted hydrocarbons (having at least N carbon atoms). In the second embodiment of the present invention, said stream may also comprise olefins having more than M carbon atoms. This stream may be recycled to the autothermal cracking step (ii).

All or a portion of the carbon monoxide and/or hydrogen separated may be used as a feed for a Fischer-Tropsch process, for example, in the second embodiment it may be used as a feed for the Fischer-Tropsch process from which the liquid hydrocarbon stream of step (i) is derived.

The process of the present invention also has the advantage that linear alpha-olefins with odd numbers of carbon atoms may be formed, such as 1-pentene and 1-heptene. In contrast, conventional LAO production by ethylene oligomerisation processes generally produces only the even numbered LAO's.

An added advantage of autothermal cracking an FT-derived feed is that the feed need not be treated to reduce the olefin content, for example by hydrotreatment or hydrocracking, prior to being fed to the autothermal cracking process. In contrast, it is conventional to treat FT-derived streams to reduce the olefins content therein prior to steam cracking because of the propensity of the olefins to cause coking in the steam cracker.

As an example, where 1-octene is the desired product, an FT reactor effluent comprising C4 to C20+ hydrocarbons is treated, for example by distillation, to remove C4 to C8 hydrocarbons, preferably to remove C4 to C9 hydrocarbons (generally referred to as a naphtha fraction), and to give a liquid hydrocarbon stream comprising linear paraffinic hydrocarbons with at least 9 carbon atoms, preferably at least 10 carbon atoms, which may be passed without treatment to reduce the olefin content to an autothermal cracking step. The C9+(C10+) liquid hydrocarbon stream obtained will autothermally crack to produce 1-octene and lower LAO's. The 1-octene and lower LAO's will be easily separable from the higher hydrocarbons in the ATC product stream which comprise unreacted components of the liquid hydrocarbon stream and cracked components which still have more than 8 carbon atoms. These higher hydrocarbons can be recycled to the ATC step.

In the process of the present invention, the ATC process may operate solely as a cracker for the liquid hydrocarbon stream i.e. in the absence of any co-fed hydrocarbons. Alternatively, hydrocarbons, such as other paraffinic hydrocarbons, may be co-fed to the ATC process.

For example, gaseous paraffinic hydrocarbons, such as ethane, propane and butane may be co-fed and will crack to produce C2 to C4 olefins, such as ethylene.

Liquid hydrocarbons, such as liquid paraffinic hydrocarbons, may also be co-fed as long as they comprise more than M, preferably at least N, carbon atoms, so that subsequent ease of product separation is not undermined.

As one particular example, a mixture of linear paraffinic hydrocarbons, where N is at least 9, which have been derived from the product stream of a Fischer-Tropsch process by separation of hydrocarbons having less than N carbon atoms therefrom and LAO's having at least N carbon atoms, where N is at least 10 may be fed to the autothermal cracking process.

The conditions in the autothermal cracking process of step (ii) are generally selected to enhance production of LAO's rather than ethylene. In particular, as would be apparent to the person skilled in the art, the production of LAO's generally requires less severe conditions than production of ethylene, for example.

Preferably, the conditions in the autothermal cracking process of step (ii) are selected to maximise production of 1-octene and/or 1-hexene.

Preferably, hydrogen is co-fed. Hydrogen co-feeds are advantageous because, in the presence of the catalyst, the hydrogen combusts preferentially relative to hydrocarbon, thereby increasing the LAO selectivity of the overall process. The amount of hydrogen combusted may be used to control the amount of heat generated and hence the severity of cracking. Thus, the molar ratio of hydrogen to oxygen can vary over any operable range provided that the ATC product stream comprising LAO's is produced. Suitably, the molar ratio of hydrogen to oxygen is in the range 0.2 to 4, preferably, in the range 0.2 to 2.

The hydrocarbon to be cracked (liquid hydrocarbon stream and any co-fed hydrocarbons) and molecular oxygen-containing gas may be contacted with the catalyst capable of supporting combustion in any suitable molar ratio, provided that the ATC product stream comprising LAO's is produced. The preferred stoichiometric ratio of hydrocarbon to oxygen-containing gas is 5 to 16, preferably, 5 to 13.5 times, preferably, 8 to 12 times the stoichiometric ratio of hydrocarbon to oxygen-containing gas required for complete combustion of the hydrocarbon to carbon dioxide and water.

The hydrocarbon is passed over the catalyst at a gas hourly space velocity of greater than 10,000 h⁻¹, preferably above 20,000 h⁻¹ and most preferably, greater than 100,000 h⁻¹. It will be understood, however, that the optimum gas hourly space velocity will depend upon the pressure.

The autothermal cracking step is usually operated at a pressure of greater than 0.5 barg. Preferably the autothermal cracking process is operated at a pressure of between 0.5-40 barg.

The severity of reaction will be reflected in the catalyst bed exit temperature and the catalyst bed exit temperature may be correspondingly lower for LAO production than for ethylene, for example.

The actual catalyst bed exit temperature may depend on a number of factors other than reaction severity, such as heat losses in the reactor, but in general, the autothermal cracking steps may suitably be carried out at catalyst bed exit temperatures in the range 600° C. to 1200° C., preferably in the range 600° C. to 1000° C. and, most preferably, in the range 650° C. to 900° C.

The oxygen-containing gas may be provided as any suitable molecular oxygen containing gas, such as molecular oxygen itself or air.

The catalyst capable of supporting combustion beyond the fuel rich limit of flammability usually comprises a Group VIII metal as its catalytic component. Suitable Group VIII metals include platinum, palladium, ruthenium, rhodium, osmium and iridium. Rhodium, and more particularly, platinum and palladium are preferred. Typical Group VIII metal loadings range from 0.01 to 100 wt %, preferably, between 0.01 to 20 wt %, and more preferably, from 0.01 to 10 wt % based on the total dry weight of the catalyst.

Where a Group VIII catalyst is employed, it is preferably employed in combination with a catalyst promoter. The promoter may be a Group IIIA, IVA, and/or VA metal. Alternatively, the promoter may be a transition metal; the transition metal promoter being a different metal to that which may be employed as the Group VIII transition metal catalytic component. Preferred promoters are selected from the group consisting of Ga, In, Sn, Ge, Ag, Au or Cu. The atomic ratio of Group VIII B metal to the catalyst promoter may be 1:0.1-50.0, preferably, 1:0.1-12.0.

Preferred examples of promoted catalysts include Pt/Ga, Pt/In, Pt/Sn, Pt/Ge, Pt/Cu, Pd/Sn, Pd/Ge, Pd/Cu, Rh/Sn, Pt/Pd/Cu and Pt/Pd/Sn catalysts.

For the avoidance of doubt, the Group VIII metal and promoter in the catalyst may be present in any form, for example, as a metal, or in the form of a metal compound, such as an oxide.

The catalyst may be unsupported, such as in the form of a metal gauze, but is preferably supported. Any suitable support may be used such as ceramic or metal supports, but ceramic supports are generally preferred. Where ceramic supports are used, the composition of the ceramic support may be any oxide or combination of oxides that is stable at high temperatures of, for example, between 600° C. and 1200° C. The support material preferably has a low thermal expansion co-efficient, and is resistant to phase separation at high temperatures.

Suitable ceramic supports include corderite, lithium aluminium silicate (LAS), alumina (α-Al₂O₃), yttria stabilised zirconia, alumina titanate, niascon, and calcium zirconyl phosphate. The ceramic supports may be wash-coated, for example, with γ-Al₂O₃.

The catalyst may be prepared by any method known in the art. For example, gel methods and wet-impregnation techniques may be employed. Typically, the support is impregnated with one or more solutions comprising the metals, dried and then calcined in air. The support may be impregnated in one or more steps. Preferably, multiple impregnation steps are employed. The support is preferably dried and calcined between each impregnation, and then subjected to a final calcination, preferably, in air. The calcined support may then be reduced, for example, by heat treatment in a hydrogen atmosphere.

Although the catalyst has been described above in terms of a single catalyst bed, the catalyst may alternatively be present as a sequential catalyst bed, as described, for example, in WO 02/04389.

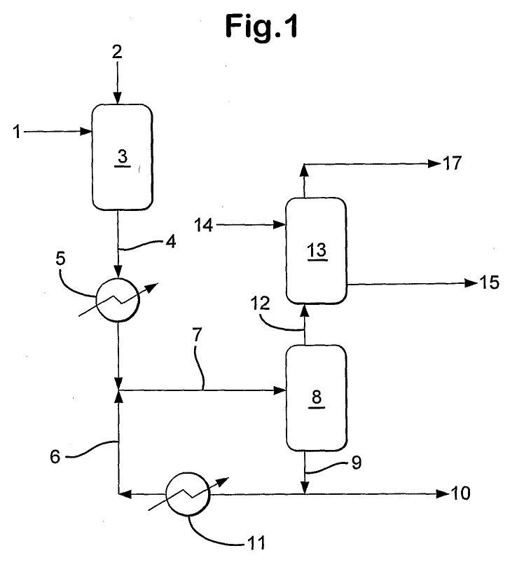

The invention will now be illustrated with respect to FIG. 1 where FIG. 1 represents in schematic form a process for the production of LAO's according to the second embodiment of the present invention.

Referring to FIG. 1, a liquid hydrocarbon stream comprising linear paraffinic hydrocarbons having at least 11 carbons atoms prepared by distillation of a full-range FT material (not shown) is provided. This stream (1) (generally referred to as FT diesel) is combined with an oxygen-containing gas (2) and fed into an autothermal cracking (ATC) reactor (3), wherein it is contacted with a catalyst capable of supporting combustion beyond the fuel-rich limit of flammability and subsequently quenched by contact with water immediately downstream of the catalyst, to produce a product stream (4) comprising LAOs having 10 and less carbon atoms.

The product stream (4) is further cooled (5), before being combined with a cooled recycle stream (6) to lower the temperature further still. The combined stream (7) is fed to a distillation column (8) wherein a bottoms stream (9) comprising C8+ hydrocarbons is separated. The bottoms stream includes LAO's, such as 1-octene and 1-decene, and heavier (unreacted) linear paraffinic hydrocarbons. A portion of this stream is recovered (10) and may be passed to further separation steps (not shown) to purify 1-octene and 1-decene, and optionally additional LAOs, such as 1-nonene. The unconverted liquid paraffinic hydrocarbons may be recycled to the ATC reactor (3) (not shown).

A further portion of this stream is cooled (11) and recycled as recycle stream (6).

The overhead stream (12) from the distillation column (8) comprises C7 and lower hydrocarbons and is passed to a water quench column (13) where water is added (14). C5-C7 hydrocarbons are removed from the base (15). This stream (15) may be passed to further separation (not shown) steps to purify 1-hexene (M=6) and, optionally, additional lower LAO's such as 1-heptene (M=7) and 1-pentene (M=5).

Non-condensable gases are recovered from the water quench column (13) as an overhead stream (17) which comprises C1-C4 hydrocarbons. Stream (17) may be submitted to additional separation steps to recover hydrogen, syn gas, fuel, and/or product olefins, such as ethylene and propylene.

Thus, a series of high value LAO's are obtained and readily separated by the process of the present invention. In particular, if the liquid hydrocarbon stream also contained lighter (e.g. FT naphtha) components such as C5-C9 linear paraffinic hydrocarbons, these components would interfere with the simple separation and purification of the produced LAO's, such as 1-octene. For example, unconverted n-octane from a non-distilled, FT-derived feedstock would be difficult to separate from the 1-octene produced from the cracking of the higher paraffins.

### EXAMPLE

This example demonstrates autothermal cracking of a liquid hydrocarbon stream comprising linear paraffinic hydrocarbons having at least N carbon atoms, where N is at least 9, which liquid hydrocarbon stream has been derived from the product stream of a Fischer-Tropsch (FT) process by separation of hydrocarbons having less than N carbon atoms therefrom, to produce an ATC product stream comprising linear alpha-olefins having M or less carbon atoms, where M is less than N.

### Catalyst Preparation—3 Wt % Platinum on Alumina.

Alumina foam blocks (10 mm diameter by 30 mm deep cylinders, 30 pores per inch) were repeatedly impregnated with an aqueous solution of tetrammineplatinum(II) chloride. The tetrammineplatinum(II) chloride solution was prepared with sufficient salt to achieve a nominal Pt loading of 3 wt % if all the metal in the salt were incorporated into the final catalyst formulation. Between impregnations excess solution was removed from the foam blocks. The foam blocks were then dried in air at 120-140° C. for approximately 30 minutes, and subsequently calcined in air at 450° C. for approximately 30 minutes (to decompose the Pt salt to Pt metal on the foam surface). Once all the solution had been absorbed onto the foams (typically three impregnations are required) the blocks were dried and given a final air calcination at 1200° C. for 6 hours.

### Hydrocarbon Feed

The liquid hydrocarbon stream for use as feed consisted of the non-hydrotreated output of an FT reactor which had been distilled to remove C8's and below and C18's and above, to produce a mixture of saturated hydrocarbons in the C9-C17 range (olefin content <2%). Analysis after distillation showed <0.01% for C8 and <0.01% for each carbon number analyzed from C18 up to C25.

### EXPERIMENTAL

Two blocks of the catalyst prepared above were located in the centre of an aluminized stainless steel reactor tube (about 10 mm ID and 13 mm OD) to form a catalyst bed. The reactor is prefitted with a quartz sleeve and alumina foam ‘heat shields’ (30 pores per inch, 10 mm diameter by 30 mm long) are placed above and below the catalyst bed. The reactor tube was located in a single-zone electrically-heated furnace and a flow of nitrogen was established upwards through the reactor and catalyst. The set point of the furnace was 600° C. and the catalyst was warmed under flowing nitrogen at about 5 nl/min. The hydrocarbon feed was run through a preheater furnace set at 425° C.

Once the reactor system has been purged of any residual air, autothermal cracking was initiated using ethane as paraffinic hydrocarbon. Once target feed conditions were achieved (ethane, hydrogen, nitrogen and oxygen feed rates of 2.39 nl/min, 0.77 nl/min, 0.44 nl/min and 0.81 nl/min respectively) the reactor was allowed to stabilize over a period of 15 minutes then a sample of the cracked gas product was taken for analysis to assure the reactor was operating properly.

At this point the ethane feed was slowly backed out and the hydrogen and oxygen flows reduced to 0.36 nl/min and 0.71 nl/min, respectively as the liquid hydrocarbon stream was introduced to a level of 4 g/min using an HPLC pump. The overall hydrocarbon feed was maintained at a rough steady state as liquid feed replaced the gaseous ethane feed.

The effluent gas was analyzed for hydrocarbons, CO, and CO2 using a combination of online and offline GC's. Carbon balances were adjusted to 100% using nitrogen as the internal standard.

The effluent from the cracking of the liquid hydrocarbon stream comprised a mix of hydrocarbons including ethylene, propylene and C4 to C8 LAO's (including C5 and C7 LAO's). The effluent also included higher hydrocarbons, including both olefins and unreacted paraffinic hydrocarbons.

1-12. (canceled)

13. A process for the production of linear alpha-olefins, which process comprises:
(i) providing a liquid hydrocarbon stream comprising hydrocarbons having at least N carbon atoms and less than 1 mol % of hydrocarbons with less than N carbon atoms, wherein
a) the liquid hydrocarbon stream comprises linear paraffinic hydrocarbons, N is at least 9, and the liquid hydrocarbon stream comprises a mixture of linear paraffinic hydrocarbons which has been derived from the product stream of a Fischer-Tropsch process by separation of hydrocarbons having less than N carbon atoms therefrom, or
b) the liquid hydrocarbon stream is an LAO-containing stream comprising LAO's having at least N carbon atoms, where N is at least 10,
(ii) autothermally cracking the liquid hydrocarbon stream to produce an ATC product stream comprising linear alpha-olefins having M or less carbon atoms, where M is less than N, and
(iii) separating the linear alpha olefins having M or less carbon atoms from the ATC product stream.

14. A process according to claim 13 wherein the ATC product stream also comprises ethylene.

15. A process according to claim 13 wherein step (i) comprises:
(i) providing a liquid hydrocarbon stream comprising a mixture of linear paraffinic hydrocarbons having at least N carbon atoms, where N is at least 9, which liquid hydrocarbon stream has been derived from the product stream of a Fischer-Tropsch process by separation of hydrocarbons having less than N carbon atoms therefrom.

16. A process as claimed in claim 15 wherein step (iii) also produces a stream comprising unreacted linear paraffinic hydrocarbons having at least N carbon atoms and olefins having more than M carbon atoms which is recycled to the autothermal cracking step (ii).

17. A process as claimed in claim 15 wherein step (iii) also comprises separating carbon monoxide and/or hydrogen, at least a portion of which is used as a feed for the Fischer-Tropsch process from which the liquid hydrocarbon stream of step (i) is derived.

18. A process according to claim 15 wherein step (i) comprises providing an FT reactor effluent comprising C4 to C20+ hydrocarbons and treating said effluent to remove hydrocarbons with less than N carbon atoms to provide said liquid hydrocarbon stream comprising a mixture of linear paraffinic hydrocarbons having at least N carbon atoms.

19. A process as claimed in claim 18 wherein the FT reactor effluent is treated to remove hydrocarbons with less than N carbon atoms by distillation.

20. A process as claimed in claim 18 wherein the FT reactor effluent is treated and subsequently passed to the autothermal cracking step without treatment to reduce the olefin content thereof.

21. A process as claimed in claim 20 wherein the liquid hydrocarbon stream is the non-hydrotreated output of an FT reactor which has been distilled to remove C8's and below and C18's and above, to produce a mixture of saturated hydrocarbons in the C9-C17 range.

22. A process as claimed in claim 15, wherein the liquid hydrocarbon stream comprises linear paraffinic hydrocarbons having at least 11 carbon atoms and the ATC product stream comprises C10 and lower linear alpha-olefins.

23. A process according to claim 13 wherein the liquid hydrocarbon stream provided in step (i) is an LAO-containing stream comprising LAO's having at least N carbon atoms, where N is at least 10.

24. A process according to claim 22 wherein the LAO-containing stream is derived from a process for the production of LAO's by oligomerisation of ethylene, and ethylene in the ATC product stream is separated and passed to the LAO production process.
